(19) United States
(12) Patent Application Publication
(10) Pub. No.: US 2004/0266842 A1
(43) Pub. Date: Dec. 30, 2004

(54) Thiazolyl substituted triazoles as alk5 inhibitors

(76) Inventors: Laramie Mary Gaster, Essex (GB); John David Harling, Herts (GB); Jag Paul Heer, Essex (GB); Thomas David Heightman, Essex (GB); Andrew Hele Payne, Essex (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/495,779

(22) PCT Filed: Nov. 14, 2002
(86) PCT No.: PCT/EP02/12892

(30) Foreign Application Priority Data
Nov. 15, 2001 (GB) 0127433.1

(51) Int. Cl. A61K 31/427; C07D 417/04

(52) U.S. Cl. 514/365; 548/200

(57) ABSTRACT

Thiazolyl substituted triazoles of formula (I)

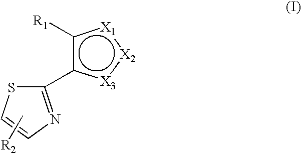

wherein R₁ is naphthyl or phenyl optionally substituted with one or more substituents selected from halo, —O—C₁₋₆alkyl, —S—C₁₋₆alkyl, C₁₋₆alkyl, C₁₋₆haloalkyl, —O—(CH₂)ₙ—Ph, —S—(CH₂)ₙ—Ph, cyano, phenyl, and CO₂R, wherein R is hydrogen or C₁₋₆alkyl, and n is 0, 1, 2 or 3; or R₁ is phenyl fused with an aromatic or non-aromatic cyclic ring of 5-7 members wherein said cyclic ring optionally contains up to three heteroatoms, independently selected from N, O and S, and N may be further optionally substituted by C₁₋₆ alkyl, and wherein the cyclic ring may be optionally substituted by ═O; or R₁ is pyridyl fused with an aromatic or non-aromatic cyclic ring of 5-7 members wherein said cyclic ring optionally contains up to three heteroatoms, independently selected from N, O and S, and N may be further optionally substituted by C₁₋₆alkyl, and wherein the cyclic ring may be optionally substituted by ═O;

R₂ is H, C₁₋₆alkyl, C₁₋₆alkoxy, phenyl, NH(CH₂)ₙ—Ph, NH—C₁₋₆alkyl, halo, CN, NO₂, CONHR and SO₂NHR;

two of X₁, X₂ and X₃ are N and the other is NR₃ wherein R₃ is hydrogen, C₁₋₆alkyl, C₃₋₇cycloalkyl, —(CH₂)ₚ—CN, —(CH₂)ₚ—CO₂H, —(CH₂)ₚ—CONHR₄R₅, —(CH)ₚCOR₄, —(CH₂)_{q}(OR₆)₂, —(CH₂)ₚOR₄, —(CH₂)_{q}CH═CH—CN, —(CH₂)_{q}—CH═CH—CO₂H, —(CH₂)ₚ—CH═CH—CONHR₄R₅, —(CH₂)ₚNHCOR₇ or —(CH₂)ₚNR₈R₉;

R₄ and R₅ are independently hydrogen or C₁₋₆alkyl;

R₆ is C₁₋₆alkyl;

R₇ is C₁₋₇alkyl, or optionally substituted aryl, heteroaryl, arylC₁₋₆alkyl or heteroarylC₁₋₆alkyl;

R₈ and R₉ are independently selected from hydrogen, C₁₋₆alkyl, aryl and arylC₁₋₆alkyl;

p is 0-4; and

q is 1-4

and salts and solvates thereof, are disclosed, as are methods for their preparation, pharmaceutical compositions containing them and their use in medicine.

This invention relates to thiazolyl substituted triazoles which are inhibitors of the transforming growth factor, (“TGF”)-β signaling pathway, in particular, the phosphorylation of smad2 or smad3 by the TGF-β type I or activin-like kinase (“ALK”)-5 receptor, methods for their preparation and their use in medicine, specifically in the treatment and prevention of a disease state mediated by this pathway.

TGF-β1 is the prototypic member of a family of cytokines including the TGF-βs, activins, inhibins, bone morphogenetic proteins and Müllerian-inhibiting substance, that signal through a family of single transmembrane serine/threonine kinase receptors. These receptors can be divided in two classes, the type I or activin lie kinase (ALK) receptors and type II receptors. The ALK receptors are distinguished from the type II receptors in that the ALK receptors (a) lack the serine/threonine rich intracellular tail, (b) possess serine/threonine kinase domains that are very homologous between type I receptors, and (c) share a common sequence motif called the GS domain, consisting of a region rich in glycine and serine residues. The GS domain is at the amino terminal end of the intracellular kinase domain and is critical for activation by the type II receptor. Several studies have shown that TGF-β signaling requires both the ALK and type II receptors. Specifically, the type R receptor phosphorylates the GS domain of the type I receptor for TGF-β, ALK5, in the presence of TGF-β. The ALK5, in turn, phosphorylates the cytoplasmic proteins smad2 and smad3 at two carboxy terminal serines. The phosphorylated smad proteins translocate into the nucleus and activate genes that contribute to the production of extracellular matrix. Therefore, preferred compounds of this invention are selective in that they inhibit the type I receptor and thus matrix production.

Activation of the TGF-β1 axis and expansion of extracellular matrix are early and persistent contributors to the development and progression of chronic renal disease and vascular disease. Border W. A., et al, N. Engl. J. Med., 1994; 331(19), 1286-92. Further, TGF-β1 plays a role in the formation of fibronectin and plasminogen activator inhibitor-1, components of sclerotic deposits, through the action of smad3 phosphorylation by the TGF-β1 receptor ALK5. Zhang Y., et al, Nature, 1998; 394(6696), 909-13; Usui T., et al, Invest. Ophthalmol. Vis. Sci., 1998; 39(11), 1981-9.

Progressive fibrosis in the kidney and cardiovascular system is a major cause of suffering and death and an important contributor to the cost of health care. TGF-β1 has been implicated in many renal fibrotic disorders. Border W. A., et al, N. Engl. J. Med., 1994; 331(19), 1286-92. TGF-β1 is elevated in acute and chronic glomerulonephritis Yoshioka K., et al, Lab. Invest., 1993; 68(2), 15463, diabetic nephropathy Yamamoto, T., et al, 1993, PNAS 90, 1814-1818., allograft rejection, HIV nepbropathy and angiotensin-induced nepbropathy Border W. A., et al, N. Engl. J. Med., 1994; 331(19), 1286-92. In these diseases the levels of TGF-β1 expression coincide with the production of extracellular matrix. Three lines of evidence suggest a causal relationship between TGF-β1 and the production of matrix. First, normal glomeruli, mesangial cells and non-renal cells can be induced to produce extracellular-matrix protein and inhibit protease activity by exogenous TGF-β1 in vitro. Second, neutralizing anti-bodies against TGF-β1 can prevent the accumulation of extracellular matrix in nephritic rats. Third, TGF-β1 transgenic mice or in vivo transfection of the TGF-β1 gene into normal rat kidneys resulted in the rapid development of glomerulosclerosis. Kopp J. B., et al, Lab. Invest., 1996; 74(6), 991-1003. Thus, inhibition of TGF-β1 activity is indicated as a therapeutic intervention in chronic renal disease.

TGF-β1 and its receptors are increased in injured blood vessels and are indicated in neointima forrnation following balloon angioplasty Saltis J., et al, Clin. Exp. Pharmacol. Physiol, 1996; 23(3), 193-200. In addition TGF-β, is a potent stimulator of smooth muscle cell (“SMC”) migration in vitro and migration of SMC in the arterial wall is a contributing factor in the pathogenesis of atherosclerosis and restenosis. Moreover, in multivariate analysis of the endothelial cell products against total cholesterol, TGF-β receptor ALK5 correlated with total cholesterol (P<0.001) Blann A. D., et al, Atherosclerosis, 1996; 120(1-2), 221-6. Furthermore, SMC derived from human atherosclerotic lesions have an increased ALK5/TGF-β type II receptor ratio. Because TGF-β1 is over-expressed in fibroproliferative vascular lesions, receptor-variant cells would be allowed to grow in a slow, but uncontrolled fashion, while overproducing extracellular matrix components McCaffrey T. A., et al, Jr., J. Clin. Invest., 1995; 96(6), 2667-75. TGF-β1 was immunolocalized to non-foamy macrophages in atherosclerotic lesions where active matrix synthesis occurs, suggesting that non-foamy macrophages may participate in modulating matrix gene expression in atherosclerotic remodeling via a TGF-β-dependent mechanism. Therefore, inhibiting the action of TGF-β1 on ALK5 is also indicated in atherosclerosis and restenosis.

TGF-β is also indicated in wound repair. Neutralizing antibodies to TGF-β1 have been used in a number of models to illustrate that inhibition of TGF-β1 signaling is beneficial in restoring function after injury by limiting excessive scar formation during the healing process. For example, neutralizing antibodies to TGF-β1 and TGF-β2 reduced scar formation and improved the cytoarchitecture of the neodermis by reducing the number of monocytes and macrophages as well as decreasing dermal fibronectin and collagen deposition in rats Shah M., J. Cell. Sci., 1995, 108, 985-1002. Moreover, TGF-β antibodies also improve healing of corneal wounds in rabbits Moller-Pedersen T., Curr. Eye Res., 1998, 17, 736-747, and accelerate wound healing of gastric ulcers in the rat, Ernst H., Gut, 1996, 39, 172-175. These data strongly suggest that limiting the activity of TGF-β would be beneficial in many tissues and suggest that any disease with chronic elevation of TGF-β would benefit by inhibiting smad2 and smad3 signaling pathways.

TGF-β is also implicated in peritoneal adhesions Saed G. M., et al, Wound Repair Regeneration, 1999 November-December, 7(6), 504-510. Therefore, inhibitors of ALK5 would be beneficial in preventing peritoneal and sub-dermal fibrotic adhesions following surgical procedures.

Surprisingly, it has now been discovered that a class of 2-thiazolyl substituted triazole compounds function as potent and selective non-peptide inhibitors of ALK5 kinase and therefore, have utility in the treatment and prevention of various disease states mediated by ALK5 linase mechanisms, such as chronic renal disease, acute renal disease, wound healing, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis and liver fibrosis, and restenosis.

Examples of diseases where fibrosis is a major component include, but are not limited to, hepatitis B virus (HBV), hepatitis C virus (HCV), alcohol-induced hepatitis, haemochromatosis and primary biliary cirrhosis.

According to the invention there is provided a compound of formula (I) or a pharmaceutically acceptable salt thereof:

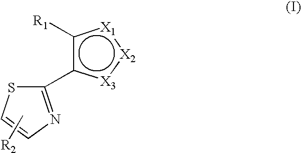

wherein R₁ is naphthyl or phenyl optionally substituted with one or more substituents selected from halo, —O—C₁₋₆alkyl, —S—C₁₋₆alkyl, C₁₋₆alkyl, C₁₋₆haloallkyl, —O—(CH₂)ₙ—Ph, —S—(CH₂)ₙ—Ph, cyano, phenyl, and CO₂R, wherein R is hydrogen or C₁₋₆allyl, and n is 0, 1, 2 or 3; or R₁ is phenyl fused with an aromatic or non-aromatic cyclic ring of 5-7 members wherein said cyclic ring optionally contains up to three heteroatoms, independently selected from N, O and S, and N may be further optionally substituted by C₁₋₆ alkyl, and wherein the cyclic ring may be optionally substituted by ═O; or R₁ is pyridyl fused with an aromatic or non-aromatic cyclic ring of 5-7 members wherein said cyclic ring optionally contains up to three heteroatoms, independently selected from N, O and S, and N may be further optionally substituted by C₁₋₆ alkyl, and wherein the cyclic ring may be optionally substituted by ═O;

R₂ is H, C₁₋₆alkyl, C₁₋₆alkoxy, phenyl, NH(CH₂)ₙ—Ph, NH—C₁₋₆alkyl, halo, CN, NO₂, CONHR and SO₂NHR;

two of X₁, X₂ and X₃ are N and the other is NR₃ wherein R₃ is hydrogen, C₁₋₆alkyl, C₃₋₇cycloalkyl, —(CH₂)ₚ—CN, —(CH₂)ₚ—CO₂H, —(CH₂)ₚ—CONHR₄R₅, —(CH₂)ₚCOR₄, —(CH₂)_{q}(OR₆)₂, —(CH₂)ₚOR₄, —(CH₂)_{q}—CH═CH—CN, —(CH₂)_{q}—CH═CH—CO₂H, —(CH₂)ₚ—CH═CH—CONHR₄R₅, —(CH₂)ₚNHCOR₇ or —(CH₂)ₚNR₈R₉;

R₄ and R₅ are independently hydrogen or C₁₋₆alkyl;

R₆ is C₁₋₆alkyl;

R₇ is C₁₋₇alkyl, or optionally substituted aryl, heteroaryl, arylC₁₋₆alkyl or heteroarylC₁₋₆alkyl;

R₈ and R₉ are independently selected from hydrogen, C₁₋₆alkyl, aryl and arylC₁₋₆alkyl;

p is 0-4; and

q is 1-4.

In the triazole ring of the compounds of formula (I) it will be apparent that the double bond will be to the two unsubstituted nitrogens.

Preferably R₁ is optionally substituted naphthyl or phenyl. More preferably R₁ is phenyl optionally substituted with one or more substituents selected from halo, C₁₋₆alkoxy, C₁₋₆alkylthio, and phenyl; or R₁ is phenyl fused with an aromatic or non-aromatic cyclic ring of 5-7 members wherein said cyclic ring optionally contains up to two heteroatoms, independently selected from N, O and S, and N may be further optionally substituted by C₁₋₆ alkyl, and wherein the cyclic ring may be optionally substituted by ═O. For example R₁ represents benzo[1,3]dioxolyl, 2,3-dihydrobenzo[1,4]dioxinyl, benzoxazolyl, benzothiazolyl, benzo[1,2,5]oxadiazolyl, benzo[1,2,5]thiadiazolyl, quinoxalinyl, dihydrobenzofiranyl, benzimidazolyl, C₁₋₆benzimidazolyl, [1,2,4]triazolo[1,5-α]pyridyl, benzo[1,4]oxazinyl-3-one, benzoxazolyl-2-one or benzo[1,4]oxazinyl. Preferably R₁ represents 4methoxyphenyl, 3-chlorophenyl, 3-fluoro-4-methoxyphenyl or 3-chloro-4-methoxyphenyl, or R₁ represents benzo[1,2,5]thiadiazolyl, [1,2,4]triazolo[1,5-α]pyridyl, dihydrobenzofuranyl, 2,3-dihydrobenzo[1,4]dioxinyl, benzimidazolyl, C₁₋₆ alkylbenzimidazolyl, benzo[1,4]oxazinyl-3-one or benzo[1,4]oxazinyl.

Preferably, R₂ is positioned meta to the point of attachment to the triazole, R₂ is preferably a methyl group.

Specific compounds of the invention which may be mentioned include the following and pharmaceutically acceptable salts thereof:

4-(4-Methoxyphenyl)-5-(4-methylthiazol-2-yl)-2H-[1,2,3]triazole;

6-[5-(4-Methylthiazol-2-yl)-1H -[1,2,3]triazol4H -yl]-4H-benzo[1,4]oxazin-3-one;

5-[5-(4-Methylthiazol-2-yl)-1H -[1,2,3]triazol-4-yl]-benzo[1,2,5]thiadiazole;

4-(3-Fluoro4-methoxyphenyl)-5-(4-methylthiazol-2-yl)2H -[1,2,3]triazole;

4-(3-Chloro-4-methoxyphenyl)-5-(4-methylthiazol-2-yl)-2H -[1,2,3]triazole;and

6-[S-(2-Methyl-thiazol-4-yl)-1H-[1,2,3]triazol-4-yl]-[1,2,4]triazolo[1,5-α]pyridine.

Suitable pharmaceutically acceptable salts of the compounds of formula (I) include, but are not limited to, salts with inorganic acids such as hydrochloride, sulfate, phosphate, diphosphate, hydrobroride, and nitrate, or salts with an organic acid such as malate, maleate, fumarate, tartrate, succinate, citrate, acetate, lactate, methanesulfonate, p-toluenesulfonate, palmitate, salicylate, and stearate.

Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Certain of the compounds of formula (I) may exist in the form of optical isomers, e.g. diastereoisomers and mixtures of isomers in all ratios, e.g. raceric mixtures. The invention includes all such forms, in particular the pure isomeric forms. The different isomeric forms may be separated or resolved one from the other by conventional methods, or any given isomer may be obtained by conventional synthetic methods or by stereospecific or asymmetric syntheses.

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the formula (I) or pharmaceutically acceptable derivative thereof.

The terms “C₁₋₆alkyl” and “C₁₋₇alkyl” as used herein whether on their own or as part of a larger group e.g. C₁₋₆alkoxy, mean a straight or branched chain radical of 1 to 6 and 1 to 7 carbon atoms respectively, unless the chain length is limited thereto, including, but not limited to methyl, ethyl, n-propyl, isopropyl, n-butyl, sec-butyl, isobutyl and tert-butyl.

C₁₋₆haloalkyl groups may contain one or more halo atoms, a particular C₁₋₆ haloalkyl group that may be mentioned is CF₃.

The terms “halo” or “halogen” are used interchangeably herein to mean radicals derived from the elements chlorine, fluorine, iodine and bromine.

The term “cycloalkyl” is used herein to mean cyclic radicals, preferably of 3 to 7 carbons, including but not limited to cyclopropyl, cyclopentyl and cyclohexyl.

The term “aryl” is used herein to mean 5- to 14-membered substituted or unsubstituted aromatic ring(s) or ring systems which may include bi- or tricyclic systems, including, but not limited to phenyl, naphthyl.

The term “ALK5 inhibitor” is used herein to mean a compound, other than inhibitory smads, e.g. smad6 and smad7, which selectively inhibits the ALK5 receptor preferentially over p38 or type II receptors.

The term “ALK5 mediated disease state” is used herein to mean any disease state which is mediated (or modulated) by ALK5, for example a disease which is modulated by the inhibition of the phosphorylation of smad 2/3 in the TGF-1β signaling pathway.

The term “ulcers” is used herein to include, but not to be limited to, diabetic ulcers, chronic ulcers, gastric ulcers, and duodenal ulcers.

The compounds of formula (I) can be prepared by art-recognized procedures from known or commercially available starting materials. If the starting materials are unavailable from a commercial source, their synthesis is described herein, or they can be prepared by procedures known in the art.

Specifically, compounds of formula (I) may be prepared as illustrated in Scheme 1.

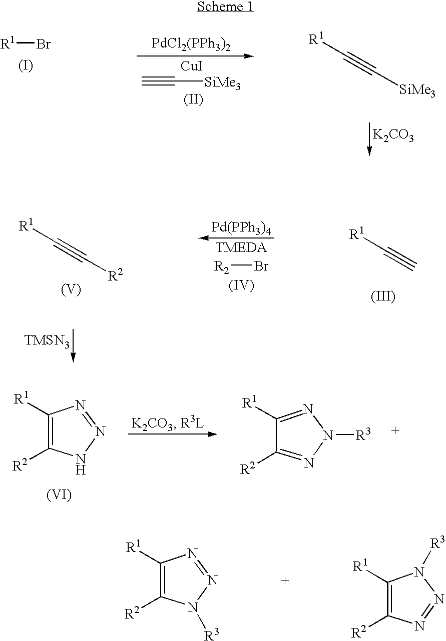

A suitable aryl bromide (I) is coupled with trimethylsilylacetylene (II) using a palladium catalyst in the presence of copper(I) iodide. The trimethylsilyl group can then removed under basic conditions with potassium carbonate and the unmasked terminal acetylene (III) coupled to an aryl bromide (IV) again via palladium catalysis. The disubstituted acetylene (V) is treated with trimethylsilylazide to afford a triazole (VI) which may be alkylated with a suitable alkylating agent, L=R³ where L is a leaving group, e.g. I, in the presence of potassium carbonate. The resulting isomers can be separated by chromatographic methods.

During the synthesis of the compounds of formula (I) labile functional groups in the intermediate compounds, e.g. hydroxy, carboxy and amino groups, may be protected. A comprehensive discussion of the ways in which various labile functional groups groups may be protected and methods for cleaving the resulting protected derivatives is given in for example Protective Groups in Organic Chemistry, T. W. Greene and P. G. M. Wuts, (Wiley-Interscience, New York, 2nd edition, 1991).

Further details for the preparation of compounds of formula (I) are found in the examples.

The compounds of formula (I) may be prepared singly or as compound libraries comprising at least 2, for example 5 to 1,000 compounds, and more preferably 10 to 100 compounds of formula (I). Libraries of compounds of formula (I) may be prepared by a combinatorial ‘split and mix’ approach or by multiple parallel synthesis using either solution phase or solid phase chemistry, by procedures known to those skilled in the art.

Thus according to a further aspect of the invention there is provided a compound library comprising at least 2 compounds of formula (I) or pharmaceutically acceptable salts thereof.

In another aspect of the invention there is provided a compound of formula (I), or a pharmaceutically acceptable salt or solvate thereof, for use in therapy.

According to a further aspect of the present invention there is provided the use of a compound of formula (I) or a pharmaceutically acceptable salt thereof, in the manufacture of a medicament for the treatment of a disease mediated by the ALK5 receptor in mammals.

ALK5-mediated disease states, include, but are not limited to, chronic renal disease, acute renal disease, wound healing, arthritis, osteoporosis, kidney disease, congestive heart failure, ulcers, ocular disorders, corneal wounds, diabetic neuropathy, impaired neurological function, Alzheimer's disease, atherosclerosis, peritoneal and sub-dermal adhesion, any disease wherein fibrosis is a major component, including, but not limited to lung fibrosis and liver fibrosis, for example, hepatitis B virus (HBV), hepatitis C virus (HCV), alcohol-induced hepatitis, haemochromatosis and primary biliary cirrhosis and restenosis.

By the term “treating” is meant either prophylactic or therapeutic therapy.

According to a further aspect of the present invention there is provided a method of inhibiting the TGF-β signaling pathway in mammals, for example, inhibiting the phosphorylation of smad2 or smad3 by the type I or activin-like kinase ALK5 receptor.

According to a further aspect of the present invention there is provided a method of inhibiting matrix formation in mammals by inhibiting the TGF-β signalling pathway, for example, inhibiting the phosphorylation of smad2 or smad3 by the type I or activin-like kinase ALK5 receptor.

The pharmaceutically effective compounds of formula (I) and pharmaceutically acceptable salts thereof, may be administered in conventional dosage forms prepared by combining a compound of formula (I) with standard pharmaceutical carriers or diluents according to conventional procedures well known in the art. These procedures may involve mixing, granulating and compressing or dissolving the ingredients as appropriate to the desired preparation.

According to a further aspect of the present invention there is provided a pharmaceutical composition comprising a compound of formula (I), or a pharmaceutically acceptable salt thereof, and a pharmaceutically acceptable carrier or diluent.

The pharmaceutical compositions of the invention may be formulated for administration by any route, and include those in a form adapted for oral, topical or parenteral administration to mammals including humans.

The compositions may be formulated for administration by any route. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-500 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 20 mg/kg per day.

It will be recognized by one of skill in the art that the optimal quantity and spacing of individual dosages of a formula (I) compound will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of the formula (I) compound given per day for a defined number of days, can be ascertained by those skilled in the art using conventional course of treatment determination tests.

No toxicological effects are indicated when a compound of formula (I) or a pharmaceutically acceptable derivative thereof is administered in the above-mentioned dosage range.

All publications, including, but not limited to, patents and patent applications cited in this specification, are herein incorporated by reference as if each individual publication were specifically and individually indicated to be incorporated by reference herein as though fully set forth.

The following examples are to be construed as merely illustrative and not a limitation on the scope of the invention in any way. In the Examples, mass spectra were performed using an Hitachi Perkin-Elmer RMU-6E with chemical ionization technique (CI) or a Micromass Platform II instrument with electrospray (ES) ionization technique.

Abbreviations

EtOAc—ethyl acetate

K₂CO₃—potassium carbonate

MgSO₄—magnesium sulphate

NaHCO₃—sodium hydrogencarbonate

Pd(PPh3)4—tetrakis(triphenylphosphine) palladium(0)

THF—tetrahydrofuran

TMEDA—N,N,N′,N′-tetramethylethyienediamine

r.t.—room temperature

Preparation 1: 2-4odo-4-methylthiazole

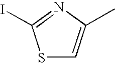

A stirred solution of 4-methylthiazole (10 g, 101.0 mmol, 1 eq) in THF (200 ml) was cooled under argon to −78° C. with dry ice/acetone. To this was added dropwise 2.5M (in hexanes) n-butyl lithium (22.5 ml, 111.1 mmol, 1.1 eq) and the mixture stirred at −78° C. for 30 minutes. A solution of iodine (28 g, 111.1 mmol, 1.1 eq) in THF (150 ml) under argon was then added to the mixture dropwise via syringe (the colour of the iodine dissipated upon stirring after each addition, but as excess iodine was added, the colour was retained). The mixture was allowed to warm to room temperature and stirred for a further 30 minutes, before being quenched with aqueous ammonium chloride solution (100 ml). The separation of organic and aqueous layers was aided by the addition of ethyl acetate (100 ml) and water (100 ml), and the organic phase was washed with saturated aqueous sodium thiosulfate solution (200 ml) before being dried over anhydrous magnesium sulfate and the solvents removed to yield a dark, crystalline solid (13 g, 57%). ¹H NMR (250 MHz, CDCl₃) δ: 6.87 (1H, s), 2.49 (3H, s).

Preparation 2: (4-Methoxyphenylethynyl)-trimethylsilane

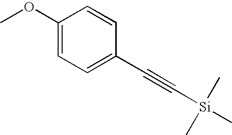

4-Bromoanisole (3 g, 16 mmol, 1 eq) was dissolved in ThF (50 ml) and TMEDA (50 ml) under argon. To this was added Pd(Ph₃)₄ (0.92 g, 0.8 mmol, 0.05 eq), copper iodide (0.31 g, 1.6 mmol, 0.1 eq) and trimethylsilylacetylene (2.7 ml, 19 mmol, 1.2 eq). The mixture was then refluxed at 60° C. for 6 h, allowed to cool, and aqueous ammonium chloride solution added (100 ml). This was then extracted with ethyl acetate (100 ml). The aqueous layer was washed with further ethyl acetate (50 ml) and the organic layers combined. The organic solution was washed with water and brine (100 ml of each), dried over anhydrous MgSO₄ and the solvent removed. Purification of the crude was carried out by flash column chromatography, eluting with 4:1 40-60° C. petroleum ether: ethyl acetate to yield a pale oil (4.2 g, 92%). ¹H NMR (250 MHz, CDCl₃) δ: 7.21 (2H, d), 6.61 (2H, d), 3.81 (3H, s), 0.1 (9H, s).

Preparation 3: 4-Ethynyl -1-methoxybenzene

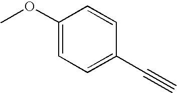

The (4-methoxyphenylethynyl)-tethylsilane (4.2 g, 24 mmol, 1 eq) was dissolved in methanol (100 ml) and to this was added potassium carbonate (10.14 g, 73 mmol, 3 eq). The suspension was stirred for 2 h and the solvent removed. The residue was suspended in water (100 ml) and extracted with ethyl acetate (2×100 ml). The organic layers were then combined, washed with water and brine (50 ml of each), dried over anhydrous MgSO₄, and the solvent removed to yield a pale oil (2.7 g, 100%). ¹H NMR (400 MHz, CDCl₃): 7.44 (2H, d), 6.84 (2H, d), 3.81 (3H, s), 1.56 (1H, s).

Preparation 4: 2(4Methoxyphenylethynyl)-4-methylthiazole

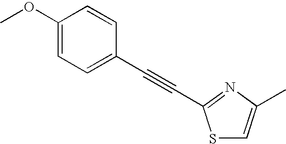

4-Ethynyl-1-methoxybenzene (0.5 g, 3.8 mmol, 1 eq) was dissolved in anhydrous THF (20 ml) and TUEDA (20 ml) under argon. To this was added Pd(PPh₃)₄ (0.22 g, 0.10 mmol, 0.05 eq), copper iodide (0.1 g, 0.38 mmol, 0.1 eq) and 2-iodo-4-methylthiazole,(1.01 g, 4.5 mmol, 1.2 eq The mixture was then refluxed at 70° C. for 48 h, allowed to cool, and aqueous ammonium chloride solution added (50 ml). This was then extracted with ethyl acetate (50 ml). The aqueous layer was washed with further ethyl acetate (20 ml) and the organic layers combined. The organic solution was washed with water and brine (20 ml of each), dried over anhydrous MgSO₄ and the solvent removed. Purification by flash chromatography over silica, eluted with 4:1 40-60° C. petroleum ether: ethyl acetate, afforded a sample of pale yellow solid (340 mg, 40%). ¹H NMR (250 MHz, CDCl₃): 7.49 (2H, d), 6.88 (2H, d), 3.83 (3H, s), 2.48 (3H, m).

Preparation 5: (4-Bromo-2-nitrophenoxy)acetic acid ethyl ester

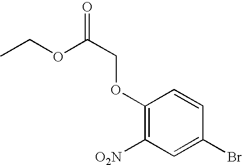

To a stirred solution of 4-bromo-2-nitrophenol (3.71 g, 17.0 mmol, 1.0 eq) in DMF (80 ml) at r.t. was added solid K₂CO₃ (4.70 g, 34.0 mmol, 2.0 eq). The mixture was heated at 40° C. for 3 h then allowed to cool to r.t. and partitioned between EtOAc and water. The aqueous phase was extracted with more EtOAc and the combined organic phase washed with water, brine and dried over MgSO₄. Concentration gave a yellow solid (5.01 g, 97%) which did not require further purification. ¹H NMR (250 MHz; CDCl₃) δ: 8.00 (1H, d), 7.62 (1H, dd), 6.90 (1H, d), 4.76 (1H, s), 4.26 (2H, q), 1.29 (3H, t).

Preparation 6: 6-Bromo-4H-benzo[1,4]oxazin-3-one

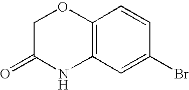

To a stirred solution of (4-bromo-2-nitrophenoxy)acetic acid ethyl ester (4.01 g, 13.2 mmol, 1.0 eq) in glacial acetic acid (70 ml) at r.t. was added iron powder (14.70 g, 264.0 mmol, 20.0 eq). The mixture was stirred vigorously at 60° C. for 4 h then allowed to cool to ambient temperature. The mixture was filtered through a pad of Kieselguhr, washing through with EtOAc, and the solution evaporated to dryness. The residue was partitioned between saturated aqueous NaHCO₃ solution and EtOAc. The aqueous was extracted with EtOAc and the combined organic phase washed with water, brine and dried over MgSO₄. Concentration gave the desired benzoxazinone (2.90 g, 97%) as a white solid which did not require purification. ¹H NMR (250 MHz; CDCl₃) δ: 10.79 (1H, br.s) 7.09-7.01 (2H, m), 6.91 (1H, d), 4.59 (2H, s).

Preparation 7: N′-(5-Bromo-2-aminopyridine)-N,N-dimethylformamidine

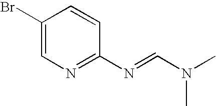

5-Bromo-2-aminopyridine (9.8 g, 56.6 mmol, 1 eq) was dissolved in dry DMF (20 ml) and dry dirnethylformamide dimethylacetal (20 ml) under argon. The solution was refluxed at 130° C. for 16 h, allowed to cool, and the solvents removed. The resultant residue was used in the next stage without purification; m/z [APCIMS]: 228.0/230.0 [M+H]⁺.

Preparation 8: 6-Bromo-[1,2,4]triazolo[1,5-α]pyridine

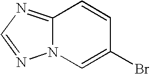

N′-(5-Bromo-2-aminopyridine)-N,N-dimethylformamidine (16.2 g, ˜56.6 mmol, 1 eq) was dissolved in methanol (90 ml) and pyridine (10 ml) under argon and cooled down to 0° C. To the reaction mixture was added, with stirring, hydroxylamine-O-sulfonic acid (7.3 g, 75.2 mmol, 1.3 eq) to form a purple suspension. The mixture was allowed to reach r.t. and stirred for 16 h. After removing the solvents, the residue was suspended in aqueous sodium hydrogen carbonate (200 ml) and extracted with EtOAc (2×200 ml). The organic layer was then washed with water and brine (100 ml of each), dried (MgSO₄) and the solvent removed. Purification by flash chromatography on silica, eluting with a gradient solvent system of first 2:1 40-60° C. petroleum ether:EtOAc to 1:1 40-60° C. petroleum ether:EtOAc afforded the product as a pale yellow solid (5 g, 44.6%); ¹H NMR (250 MHz; CDCl₃) δ: 8.77 (1H, s), 8.34 (1H, s), 7.69 (1H, d), 7.65 (1H, d); m/z [APCIMS]: 198.0/200.0 [M+H]⁺.

Preparation 9: 6-Trimethylsilanylethynyl-[1,2,4]triazolo[1,5-α]pyridine

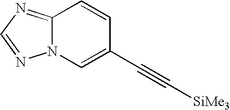

6-Bromo-[1,2,4]triazolo[1,5-α]pyridine (5 g, 25.26 mmol, 1 eq) was dissolved in THF (50 ml) and argon bubbled through the solution for 5 min. To this was added copper iodide (0.46 g, 2.53 mmol, 0.1 eq), dichlorobistriphenylphosphine (0.36 g, 0.51 mmol, 0.02 eq), and trimethylsilylacetylene (7.14 ml, 4.96 g, 50.52 mmol, 2 eq). Diisopropylamine (6.78 ml, 5.1 g, 50.52 mmol, 2 eq) was added dropwise to the solution and the resulting deep red suspension stirred under argon for 24 h. The suspension was then filtered through celite, washing with an excess of EtOAc, and the solvents removed. The residue was then suspended in water (200 ml) and extracted with EtOAc (2×200 ml), and the organic layers combined, washed with water and brine (100 ml of each), dried (MgSO₄), and the solvent removed. Purification by flash chromatography over silica, eluting with 3:1 40-60° C. petroleum ether: EtOAc afforded the product as a pale yellow solid (2.9 g, 53.3%); ¹H NMR (400 MHz; CDCl₃) δ: 8.72 (1H, s), 8.36 (1H, s), 7.69 (1H, d), 7.54, (1H, d), 0.28 (9H, s); m/z [APCIMS]: 216 [M+H]⁺.

Preparation 10: 6-Ethynyl-[1,2,4]triazolo[1,5-α]pyridine

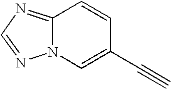

6-Trimethylsilanylethynyl-[1,2,4]triazolo[1,5-α]pyridine (2.9 g, 13.47 mmol, 1 eq) was dissolved in methanol and to this was added potassium carbonate (5.6 g, 40.4 mmol, 3 eq). The suspension was stirred for 2 h and the solvent removed. The residue was suspended in water (100 ml) and extracted with ethyl acetate (2×100 m). The organic layers were then combined, washed with water and brine (50 ml of each), dried (MgSO₄), and the solvent removed to give a pale orange solid (1.8 g, 95%) that was used in the next reaction without farther purification; m/z [APCIMS]: 144.1 [M+H]⁺.

Preparation 11: 6(2-Methyl-thiazol-4-ylethynyl)-[1,2,4]triazolo[1,5-α]pyridine

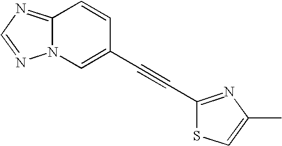

A stirred solution of 6-ethynyl-[1,2,4]triazolo[1,5-α]pyridine (750 mg, 5.245 mmol) in TMEDA (20 ml) and THF (20 ml) was degassed with argon, treated with Pd(PPh₃)₄ (275 mg, 0.238 mmol), CuI (109 mg, 0.574 mmol) and 2-iodo-4-methylthiazole (2.385 g, 10.6 mmol), and heated at 50° C. for 16 hours under argon. The solvent was removed in vacuo and the residue partioned between EtOAc (3×70 ml) and sat. aq. NaHCO₃ (70 ml). The ethyl acetate layers were combined, dried over Na₂SO₄, filtered and concentrated to dryness in vacuo. The residue was purified using silica gel chromatography, eluting with 2:1 EtOAc/petroleum ether to give a pale yellow crystalline solid. (1.12 g, 895%). CIMS: 241 M+H]⁺.

### EXAMPLES

### Example 1

### 4-(4-Methoxyphenyl)-5-(4-methylthiazol-2-yl)-2H-[1,2,3]triazole

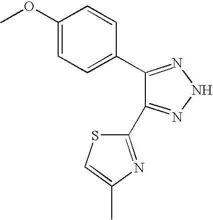

2-(4-Methoxyphenylethynyl)-4-methylthiazole (250 mg, 1.1 mmol, 1 eq) was dissolved in anhydrous DMF (1.5 ml) in the reaction vessel, and argon bubbled through for 5 minutes. To this was added trimethylsilylacetylene azide (0.5 ml, 4 eq) and more argon bubbled through. After this was complete, the vessel was sealed under argon and heated to 100° C. for 48 h. After cooling, the mixture was taken up in water (30 ml) and extracted with ethyl acetate (2×30 ml). The organic layers were combined, washed with water and brine (20 ml of each), dried over anhydrous MgSO₄ and the solvent removed. The crude was then purified by flash column chromatography, eluting with 3:1 40-60° C. petroleum ether: ethyl acetate to yield a yellow solid product (100 mg, 32%). ¹H NMR (250 MHz, CDCl₃) 6: 7.91 (2H, d), 6.96 (2H, d), 3.88 (3H, s), 2.51 (3H, s), NH not observed.

### Example 2

### 6-[5-(4-Methylthiazol-2-yl)-1H-[1,2,3]triazolyl]-4H-benzo[1,4]oxazin-3-one

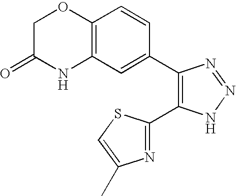

Prepared from 6-(4-methylthiazol-2-ylethynyl)-4H-benzo[1,4]oxazin-3-one using a method similar to that described for Example 1. ¹H NMR (250 MHz; DMSO-d⁶) δ: 10.88 (1H, br.s), 7.70-7.50 (2H, br.m), 7.31 (1H, s), 7.03 (1H, d), 4.64 (2H, s), 2.43 (3H, s), triazole NH not observed; m/z [ESMS]: 314.1 [M+H]⁺.

### Example 3

### 5-[5-(4-Methylthiazol-2-yl)-1H-[1,2,3]triazol-4-yl]-benzo[1,2,5]thiadiazole

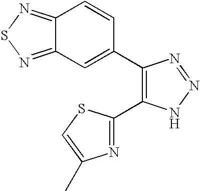

Prepared from 5-(4-methylthiazol-2-ylethynyl)benzo[1,2,5]thiadiazole using a method similar to that described for Example 1. ¹H NMR (250 MHz, CDCl₃) δ: 8.8 (1H, s), 8.2 (1H, d, J=9 Hz), 8.0 (1H, d, J=9 Hz), 7.0 (1H, s), 2.5 (3H, s); m/z (API⁺): 301 (MH⁺), NH not observed.

### Example 4

### 4-(3-Fluoromethoxyphenyl)-5-(4-methylthiazol-2-yl)-2H-[1,2,3]triazole

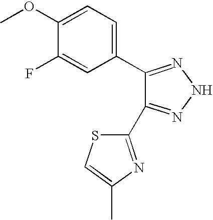

Prepared from 2-(3-fluoro-4-methoxyphenylethynyl)-4-methylthiazole using a method similar to that described for Example 1. ¹H NMR (250 MHz, CDCl₃) δ: 7.85 (1H, dd), 7.70 (1H, d), 6.97 (2H, m), 3.92 (3H, s), 2.52 (3H, s), NH not observed.

### Example 5

### 4-(3-Chloro-4-methoxyphenyl)-5-(4-methylthiazol-2-yl)-2H-[1,2,3]triazole

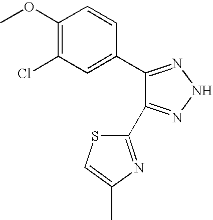

Prepared from 2-(3-chloro-4-methoxyphenylethynyl)-4-methylibiazole using a method similar to that described for Example 1. ¹H NMR (400 MHz, CDCl₃) δ: 8.20 (1H, d), 7.92 (1H, dd), 7.03 (1H, d), 6.95 (1H, s), 3.96 (3H, s), 2.51 (3H, s), NH not observed; m/z [APCIMS]: 305.0 [M−H⁺]

### Example 6

### 6-[5-(2-Methyl-thiazol-4yl)-1H-[1,2,3]triazol-4-yl]-[1,2,4]triazolo[1,5-α]pyridine

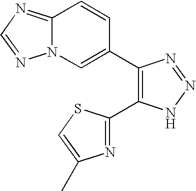

A stirred solution of 6-(2-methyl-thiazol-4-ylethynyl)-[1,2,4]triazolo[1,5-α]pyridine ( 560 mg, 2.3mmol) in DMF (3 ml) under argon was treated with azidotiethylsilane (0.92 ml, 7 mmol), and heated at 130° C. for 19 hours. The DMF was removed in vacuo and the residue partitioned between ethyl acetate (2×25 ml) and water (50 ml). The combined organic layers were extracted with aq. sodium hydroxide (2 M) (2×25 ml). The combined sodium hydroxide layers were neutralised with aq. HCl (2 M) and extracted with EtOAc (50 ml). The organic layer was dried over Na₂SO₄, and evaporated to dryness. Silica chromatography eluting with EtOAc/petroleum ether (1:1→8:2→neat EtOAc) gave an off white solid (34 mg, 5%). A solution in methanol was treated with ethereal HCl (1 M, 120 ul), and concentrated to dryness giving a pale yellow solid (38 mg).

m/z [ESMS]: 282 [M−H]⁻. 1H NMR (HCl salt, 400 MHz; DMSO-d⁶) δ: 15.80 (1H, br. s), 10.28 (1H, br. s), 8.60 (1H, s), 8.25 (1H, d), 7.97 (H,d), 7.46 (1H, s), 2.50 (3H, s); NHs not observed.

Biological Data

The biological activity of the compounds of the invention may be assessed using the following assays:

Fluorescence Anisotropy Kinase Binding Assay

The kinase enzyme, fluorescent ligand and a variable concentration of test compound are incubated together to reach thermodynamic equilibrium under conditions such that in the absence of test compound the fluorescent ligand is significantly (>50%) enzyme bound and in the presence of a sufficient concentration (>10×Kᵢ) of a potent inhibitor the anisotropy of the unbound fluorescent ligand is measurably different from the bound value.

The concentration of kinase enzyme should preferably be ≧1×K_{f}. The concentration of fluorescent ligand required will depend on the instrumentation used, and the fluorescent and physicochemical properties. The concentration used must be lower than the concentration of kinase enzyme, and preferably less than half the kinase enzyme concentration. A typical protocol is:

All components dissolved in Buffer of final composition 50 m HEPES, pH 7.5, 1 mM CHAPS, 1 mM DTT, 10 mM MgCl₂ 2.5% DMSO.

ALK5 Enzyme concentration: 4 nM

Fluorescent ligand concentration: 1 nM

Test compound concentration: 0.1 nM-100 uM

Components incubated in 10 ul final volume in LJL HE 384 type B black microtitre plate until equilibrium reached (5-30 mins)

Fluorescence anisotropy read in LJL Acquest.

Definitions: Kᵢ=dissociation constant for inhibitor binding

K_{f}=dissociation constant for fluorescent ligand binding

The fluorescent ligand is the following compound:

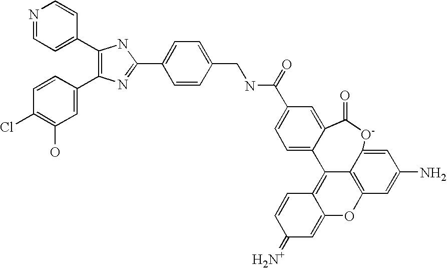

which is derived from 5-[2-4-aminomethylphenyl)-5-pyidin-4-yl-1H-inidazolyl]-2-chlorophenol and rhodamine green.

The compounds of this invention generally show ALKS receptor modulator activity having IC₅₀ values in the range of 0.0001 to 10 μM.

1. A compound of formula (I) or a pharmaceutically acceptable salt thereof: 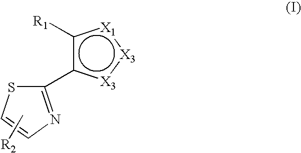
wherein R₁ is naphthyl or phenyl optionally substituted with one or more substituents selected from halo, —O—C₁₋₆alkyl, —S—C₁₋₆alkyl, C₁₋₆alkyl, C₁₋₆haloalkyl, —O—(CH₂)—Ph, —S—(CH₂)ₙ—Ph, cyano, phenyl, and CO₂R, wherein R is hydrogen or C₁₋₆alkyl, and n is 0, 1, 2 or 3; or R₁ is phenyl fused with an aromatic or non-aromatic cyclic ring of 5-7 members wherein said cyclic ring optionally contains up to three heteroatoms, independently selected from N, O and S, N may be further optionally substituted by C₁₋₆ alkyl, and wherein the cyclic ring may be optionally substituted by ═O; or R₁ is pyridyl fused with an aromatic or non-aromatic cyclic ring of 5-7 members wherein said cyclic ring optionally contains up to three heteroatoms, independently selected from N, O and S, and N may be further optionally substituted by C₁₋₆alkyl, and wherein the cyclic ring may be optionally substituted by ═O;
R₂ is H, C₁₋₆alkyl, C₁₋₆alkoxy, phenyl, NH(CH₂)ₙ—Ph, NH—C₁₋₆alkyl, halo, CN, NO₂, CONHR and SO₂NHR;
two of X₁, X₂ and X₃ are N and the other is NR₃ wherein R₃ is hydrogen, C₁₋₆alkyl, C₃₋₇cycloalkyl, —(CH₂)ₚ—CN, —(CH₂)ₚ—CO₂H, —(CH₂)ₚ—CONHR₄R₅, —(CH₂)ₚCOR₄, —(CH₂)_{q}(OR₆)₂, —(CH₂)ₚOR₄, —(CH)_{q}—CH═CH—CN, —(CH₂)_{q}—CH═CH—CO₂H, —(CH₂)ₚ—CH═CH—CONHR₄R₅, —(CH₂)ₚNHCOR₇ or —(CH₂)ₚNR₈R₉;
R₄ and R₅ are independently hydrogen or C₁₋₆alkyl;
R₆ is C₁₋₆alkyl;
R₇ is C₁₋₇alkyl, or optionally substituted aryl, heteroaryl, arylC₁₋₆alkyl or heteroarylC₁₋₆alkyl;
R₈ and R₉ are independently selected from hydrogen, C₁₋₆alkyl, aryl and arylC₁₋₆alkyl;
p is 0-4; and
q is 1-4.

2. A compound according to claim 1 wherein R₁ is phenyl optionally substituted with one or more substituents selected from halo, C₁₋₆alkoxy, C₁₋₆alkylthio, and phenyl; or R₁ is phenyl fused with an aromatic or non-aromatic cyclic ring of 5-7 members wherein said cyclic ring optionally contains up to two heteroatoms, independently selected from N, O and S, and N may be further optionally substituted by C₁₋₆ alkyl, and wherein the cyclic ring may be optionally substituted by =0.

3. A compound according to claim 1 wherein R₁ is pyridyl optionally substituted with one or more substituents selected from halo, C₁₋₆alkoxy, C₁₋₆alkylthio, and phenyl; or R₁ is phenyl fused with an aromatic or non-aromatic cyclic ring of 5-7 members wherein said cyclic ring optionally contains up to two heteroatoms, independently selected from N, O and S, and N may be further optionally substituted by C₁₋₆alkyl, and wherein the cyclic ring may be optionally substituted by =0.

4. A compound according to or claim 3 wherein R₁ represents 4-methoxyphenyl, 3-chlorophenyl, 3-fluoro-4-methoxyphenyl or 3-chloro-4-methoxyphenyl, or R₁ represents benzo[1,2,5]thiadiazolyl, [1,2,4]triazolo[1,5-α]pyridyl, dihydrobenzofuranyl, 2,3-dihydrobenzo[1,4]dioxinyl, benzimidazolyl, C₁₋₆benzimidazolyl, benzo[1,4]oxazinyl-3-one or benzo[1,4]oxazinyl.

5. A compound according to claim 1 wherein R₂ is positioned meta to the point of attachment to the triazole.

6. A compound according to claim 5 wherein R₂ is methyl.

7. A compound according to claim 1 selected from:
4-(4-Methoxyphenyl)-5-(4-methylthiazol-2-yl)-2H-[1,2,3]triazole;
6-[5-(4-Methylthiazol-2-yl)-1H-[1,2,3]triazol-4-yl]-4H-benzo[1,4]oxazin-3-one;
5-[5-(4-Methylthiazol-2-yl)-1H-[1,2,3]triazol-4-yl]-benzo[1,2,5]thiadiazole;
4-(3-Fluoro-4-methoxyphenyl)-5-(4-methylthiazol-2-yl)2H- [1,2,3]triazole;
4-(3-Chloro-4-methoxyphenyl)-5-(4-methylthiazol-2-yl)-2H-[1,2,3]triazole; and
6-[5-(2-Methyl-thiazol-4-yl)-1H-[1,2,3]triazol-4-yl]-[1,2,4]triazolo[1,5-α pyridine;
and pharmaceutically acceptable salts thereof.

8. A pharmaceutical composition comprising a compound according to claim 1, or a pharmaceutically acceptable salt thereof, and a pharmaceutically acceptable carrier or diluent.

9. A compound of formula (I) as claimed in claim 1, or a pharmaceutically acceptable salt or solvate thereof, for use in therapy.

10. (Cancelled)

11. A method of inhibiting the TGF-β signaling pathway in mammals, comprising administering to a mammal in need of such treatment, a therapeutically effective amount of a compound according to claim 1, or a pharmaceutically acceptable salt thereof.

12. A method for treating a disease selected from chronic renal disease, acute renal disease, wound healing, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis and liver fibrosis, for example, hepatitis B virus (HBV), hepatitis C virus (HCV), alcohol-induced hepatitis, haemochromatosis and primary biliary cirrhosis and restenosis, comprising administering to a mammal in need of such treatment, a therapeutically effective amount of a compound according to claim 1, or a pharmaceutically acceptable salt thereof.

13. A method for inhibiting matrix formation in mammals, comprising administering to a mammal, a therapeutically effective amount of a compound according to claim 1, or a pharmaceutically acceptable salt thereof.
